(19) United States
(12) Patent Application Publication
(10) Pub. No.: US 2018/0263599 A1
(43) Pub. Date: Sep. 20, 2018

(54) ULTRASONIC PROBE AND ULTRASONIC DIAGNOSTIC APPARATUS

(71) Applicant: KONICA MINOLTA, INC., Chiyoda-ku Tokyo (JP)

(72) Inventor: Koji OURA, Midori-ku Yokohama-shi Kanagawa (JP)

(21) Appl. No.: 15/922,130

(22) Filed: Mar. 15, 2018

(30) Foreign Application Priority Data
Mar. 16, 2017 (JP) 2017-051265

(51) Int. Cl. A61B 8/00; B06B 1/06; A61B 8/08

(52) U.S. Cl. CPC A61B 8/4455; A61B 8/4488; A61B 8/483; A61B 8/4461; B06B 1/064

(57) ABSTRACT

An ultrasonic probe includes: a support base; a rotation body which rotates while being supported by the support base; an ultrasonic oscillator which is disposed on an outer peripheral surface of the rotation body; and a rotary electric connector which is disposed to axially support the rotation body by the support base and exchanges electric signals with the ultrasonic oscillator, wherein the rotary electric connector includes: a rotary electrode which is bonded to the rotation body and rotates along with the rotation body, a fixed electrode which is bonded to the support base to face the rotary electrode and forms an annular guide groove in a rotation axis rotation in a region facing the rotary electrode, and a rotary contact member that is disposed inside the guide groove and rolls along the guide groove while contacting both the rotary electrode and the fixed electrode.

### CROSS-REFERENCE TO RELATED APPLICATIONS

The present application claims priority under 35 U.S.C. § 119 to Japanese Patent Application No. 2017-051265, filed on Mar. 16, 2017, the entire disclosure of which are incorporated herein by reference.

### BACKGROUND

### Technological Field

The present disclosure relates to an ultrasonic probe and an ultrasonic diagnostic apparatus.

### Description of the Related Art

An ultrasonic probe capable of oscillating an ultrasonic oscillator using a motor is known (for example, see JP 2013-048802 A).

In general, an ultrasonic diagnostic apparatus transmits an ultrasonic beam from an ultrasonic oscillator disposed in an ultrasonic probe to a subject and forms an ultrasonic image based on receiving signals which are ultrasonic echoes. Then, when the ultrasonic oscillator is oscillated by using the motor as described above, a three-dimensional ultrasonic image can be generated or a two-dimensional ultrasonic image can be generated at various angles or positions.

FIG. 1 is a diagram showing a configuration of an ultrasonic probe P according to the related art.

A distal end of the ultrasonic probe P is liquid-tightly filled with a coupling liquid which is an audio transfer medium and an ultrasonic oscillator P11 is disposed in the coupling liquid. Further, the ultrasonic oscillator P11 is attached to a rotation body P14 driven by a motor or the like in order to scan an interest region. Then, at the time of generating an ultrasonic image, the ultrasonic oscillator P11 exchanges electric signals (for example, a receiving signal of an ultrasonic echo or a drive signal for transmitting an ultrasonic beam) with a main body of an ultrasonic diagnostic apparatus while rotating together with the rotation body P14.

In the ultrasonic probe P according to the related art, as a configuration for exchanging electric signals between the ultrasonic oscillator P11 and a main body (not illustrated) of the ultrasonic diagnostic apparatus, a slip ring P1 rotating together with the rotation body P14 and a brush P2 fixed to a support body P15 are used.

More specifically, the brush P2 comes into slidable contact with the slip ring P1 and electric signals from the ultrasonic oscillator P11 are transmitted to the outside through the slip ring P1 and the brush P2. In other words, since the brush P2 and the slip ring P1 come into slidable contact with each other at all times, electric signals can be exchanged between the ultrasonic oscillator P11 and the main body.

However, there is a case in which a slidable contact between the brush P2 and the slip ring P1 breaks momentarily. In this case, since the electric signals are not exchanged between the brush P2 and the slip ring P1, for example, noises are superimposed on the receiving signals of the ultrasonic echo. Particularly, since the brush P2 and the slip ring P1 are disposed while being immersed into the coupling liquid, the coupling liquid flows into the slidable contact in response to the rotation of the rotation body P14 so that a contact error may easily occur.

In addition, since the ultrasonic probe P according to the related art is used in a slipping manner while the brush P2 is pressed against the slip ring P1, the brush P2 or the slip ring P1 is abraded at the slidable contact between the brush P2 and the slip ring P1. As a result, since contact resistance changes with time, there is concern that electric signals cannot be reliably exchanged between the ultrasonic oscillator P11 and the main body.

### SUMMARY

The present disclosure has been made in view of the above-described problems, and an object of the present disclosure is to provide an ultrasonic probe and an ultrasonic diagnostic apparatus capable of more reliably exchanging electric signals with an ultrasonic oscillator while rotating the ultrasonic oscillator.

To achieve the abovementioned object, according to an aspect of the present invention, an ultrasonic probe reflecting one aspect of the present invention comprises:

a support base;

a rotation body which rotates while being supported by the support base;

an ultrasonic oscillator which is disposed on an outer peripheral surface of the rotation body; and

a rotary electric connector which is disposed to axially support the rotation body by the support base and exchanges electric signals with the ultrasonic oscillator,

wherein the rotary electric connector includes:
a rotary electrode which is bonded to the rotation body and rotates along with the rotation body,
a fixed electrode which is bonded to the support base to face the rotary electrode and forms an annular guide groove in a rotation axis rotation in a region facing the rotary electrode, and
a rotary contact member that is disposed inside the guide groove and rolls along the guide groove while contacting both the rotary electrode and the fixed electrode in response to the rotation of the rotary electrode.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is a diagram showing an example of a configuration of an ultrasonic probe according to the related art;

FIG. 2 is a diagram showing an appearance of an ultrasonic diagnostic apparatus according to a first embodiment;

FIG. 3 is a block diagram showing an example of an overall configuration of the ultrasonic diagnostic apparatus according to the first embodiment;

FIG. 4 is a diagram showing an example of a configuration of an ultrasonic probe according to the first embodiment;

FIG. 5 is a side view showing a rotary electric connector according to the first embodiment;

FIG. 6 is a cross-sectional view showing the rotary electric connector according to the first embodiment;

FIG. 7 is a diagram showing a rotary contact member of the rotary electric connector according to the first embodiment;

FIG. 8 is a side view showing a rotary electric connector according to a second embodiment;

FIG. 9 is a cross-sectional view showing the rotary electric connector according to the second embodiment;

FIG. 10 is a side view showing a rotary electric connector according to a third embodiment;

FIG. 11 is a cross-sectional view showing the rotary electric connector according to the third embodiment;

FIG. 12 is an exploded diagram showing the rotary electric connector according to the third embodiment;

FIG. 13 is a diagram showing an example of a configuration of an ultrasonic probe according to a fourth embodiment;

FIG. 14 is a side view showing a rotary electric connector according to the fourth embodiment;

FIG. 15 is a cross-sectional view showing the rotary electric connector according to the fourth embodiment;

FIG. 16 is a side view showing a rotary electric connector according to a fifth embodiment; and

FIG. 17 is a cross-sectional view showing the rotary electric connector according to the fifth embodiment.

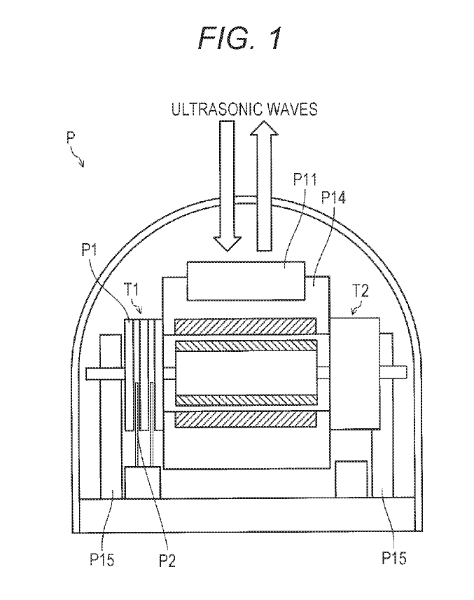

### DETAILED DESCRIPTION OF EMBODIMENTS

Hereinafter, one or more embodiments of the present invention will be described with reference to the drawings. However, the scope of the invention is not limited to the disclosed embodiments.

### First Embodiment

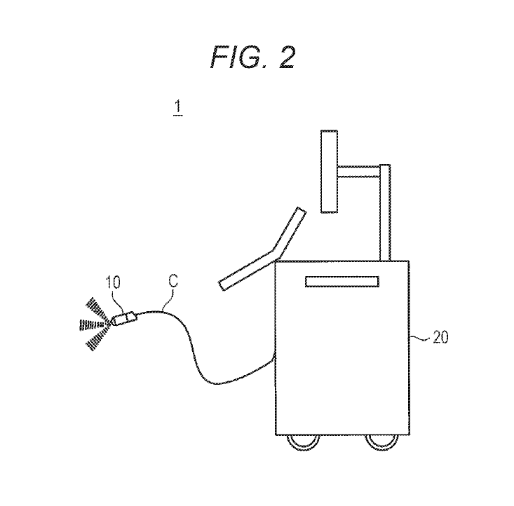

Hereinafter, a configuration of an ultrasonic probe 10 according to a first embodiment will be described with reference to FIG. 2 to FIG. 6. For example, as described above, the ultrasonic probe 10 according to the embodiment is applied to an ultrasonic diagnostic apparatus 1.

[Configuration of Ultrasonic Diagnostic Apparatus]

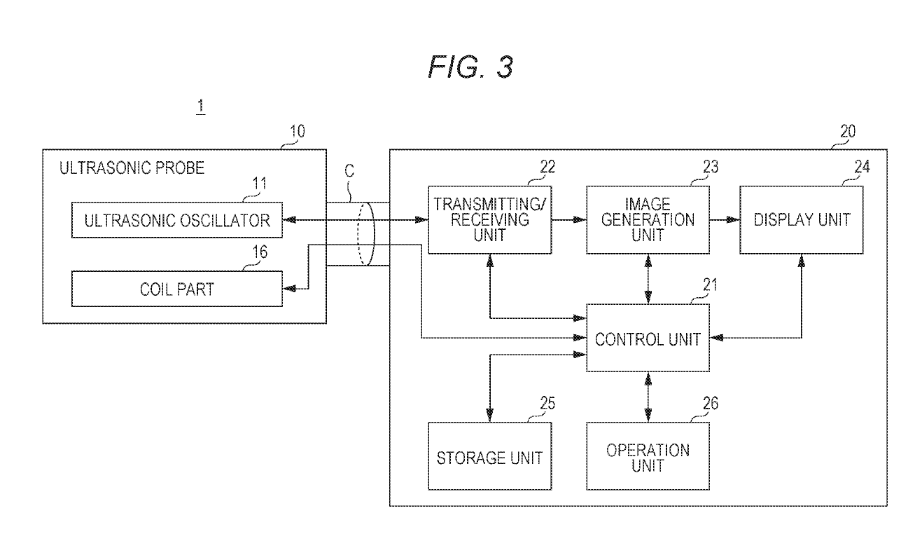

FIG. 2 is a diagram showing an appearance of the ultrasonic diagnostic apparatus 1 according to the embodiment. FIG. 3 is a block diagram showing an example of an overall configuration of the ultrasonic diagnostic apparatus 1 according to the embodiment.

The ultrasonic diagnostic apparatus 1 according to the embodiment has a configuration in which the ultrasonic probe 10 is attached to a main body 20 of the ultrasonic diagnostic apparatus 1. Further, the main body 20 and the ultrasonic probe 10 are electrically connected to each other via a cable C.

Further, the ultrasonic diagnostic apparatus 1 according to the embodiment may generate an arbitrary ultrasonic image such as a B-mode image, a color Doppler image, a three-dimensional ultrasonic image, or an M-mode image. Similarly, the ultrasonic probe 10 may be arbitrarily configured as a convex probe, a linear probe, a sector probe, or a three-dimensional probe.

The main body 20 of the ultrasonic diagnostic apparatus 1 includes a control unit 21, a transmitting/receiving unit 22, an image generation unit 23, a display unit 24, a storage unit 25, and an operation unit 26. Further, the ultrasonic probe 10 includes an ultrasonic oscillator 11, a coil part 16, and the like (a detailed configuration of the ultrasonic probe 10 will be described below).

The transmitting/receiving unit 22 is a drive circuit which transmits and receives ultrasonic waves by the ultrasonic oscillator 11 of the ultrasonic probe 10. The transmitting/receiving unit 22 transmits a voltage pulse which is a drive signal to the ultrasonic oscillator 11 and receives an electric signal relating to an ultrasonic echo generated by the ultrasonic oscillator 11 under the control of the control unit 21.

The image generation unit 23 generates an ultrasonic image (for example, a B-mode image, a color Doppler image, and a three-dimensional ultrasonic image) by performing predetermined signal processes (a logarithmic compression process, a detection process, an FFT analysis process, and the like) on the signal acquired from the transmitting/receiving unit 22. Further, since the contents of the process for generating the ultrasonic image are known, a description thereof will be omitted.

The display unit 24 is, for example, a liquid crystal display or the like and displays the ultrasonic image generated by the image generation unit 23.

The storage unit 25 is, for example, a memory such as a hard disk, a ROM, and a RAM and stores a control program or various data referred by the control unit 21 or image data generated by the image generation unit 23.

The operation unit 26 is, for example, a keyboard or a mouse and acquires an operation signal input by an operator.

The control unit 21 generally controls all units by communicating with the units of the ultrasonic diagnostic apparatus 1.

The control unit 21 controls, for example, the transmitting/receiving unit 22 and transmits and receives ultrasonic waves by the ultrasonic oscillator 11. Further, the control unit 21 controls a drive motor by generating a three-phase AC current using an embedded inverter circuit or the like and supplying the current to the coil part 16 to rotate the ultrasonic oscillator 11. In addition, the control unit 21 includes, for example, a central processing unit (CPU), a read only memory (ROM), and a random access memory (RAM) and performs each process under the control of a program.

Further, the main body 20 and the ultrasonic probe 10 of the ultrasonic diagnostic apparatus 1 are connected to each other via a cable C accommodating various wires therein. The cable C according to the embodiment accommodates a signal wire and a ground wire connected to the ultrasonic oscillator 11, an electric control wire supplying electric power to the coil part 16, and a signal wire transmitting a sensor signal of an encoder or the like and is connected to the main body 20.

[Configuration of Ultrasonic Probe]

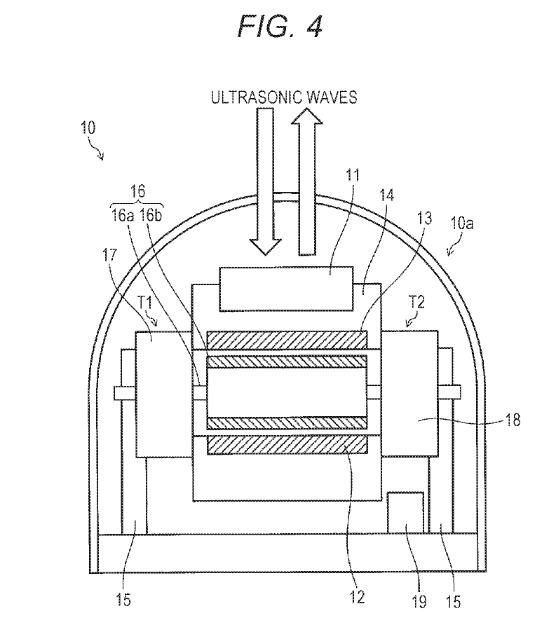

FIG. 4 is a diagram showing an example of a configuration of the ultrasonic probe 10 according to the embodiment. FIG. 4 shows a window portion 10a at a distal end of the ultrasonic probe 10.

The ultrasonic probe 10 includes the ultrasonic oscillator 11, permanent magnets 12 and 13, a rotation body 14, a support base 15, the coil part 16, rotary electric connectors 17 and 18, and a sensor unit 19 and accommodates these in a casing.

The window portion 10a of the ultrasonic probe 10 is attached to the distal end and keeps its inner space filled with a coupling liquid which is an audio transfer medium. Then, the ultrasonic oscillator 11 and the like (the ultrasonic oscillator 11, the permanent magnets 12 and 13, the rotation body 14, the support base 15, the coil part 16, the rotary electric connectors 17 and 18, and the sensor unit 19) are disposed inside the window portion 10a while being immersed into the coupling liquid. Further, a front surface of the window portion 10a is formed of a material that transmits ultrasonic waves in order to transmit and receive ultrasonic waves by the ultrasonic oscillator 11.

The ultrasonic oscillator 11 is a simple transducer or transducer array and includes, for example, one or a plurality of piezoelectric elements. The ultrasonic oscillator 11 converts a voltage pulse into an ultrasonic beam and transmits the ultrasonic beam to a subject. At the same time, an ultrasonic echo reflected inside the subject is acquired and is converted into an electric signal. Accordingly, the ultrasonic oscillator 11 performs such an operation while rotating along with the rotation body 14 and scans a target region inside the subject.

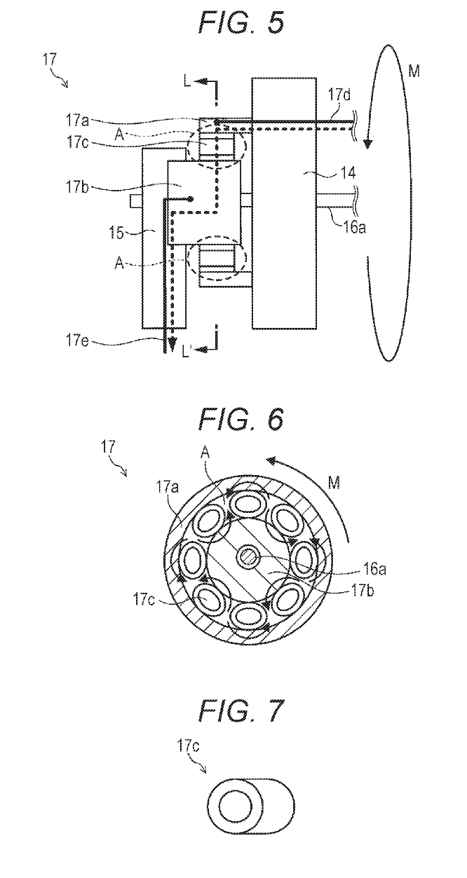

A signal wire 17d (see FIG. 5) and a ground wire (not illustrated) are connected to the ultrasonic oscillator 11 and the ultrasonic oscillator 11 exchanges electric signals with the transmitting/receiving unit 22 of the main body 20 via the signal wire 17d and is ground-connected to the main body 20 via a ground wire.

Further, the other end of the signal wire 17d connected to the ultrasonic oscillator 11 is connected to the rotary electrode of the rotary electric connector 17. Then, the ultrasonic oscillator 11 exchanges electric signals with the main body 20 via a signal wire, a rotary electric connector 17, and a lead wire from the rotary electric connector 17. Further, the other end of the ground wire connected to the ultrasonic oscillator 11 is connected to the rotary electrode of the rotary electric connector 18. Then, the ultrasonic oscillator 11 is ground-connected to the main body 20 via a ground wire, the rotary electric connector 18, and a lead wire from the rotary electric connector 18 (described later with reference to FIG. 5).

The ultrasonic oscillator 11 is disposed on the outer peripheral surface of the rotation body 14 and rotates along with the rotation body 14.

The rotation body 14 is rotated while being supported by the support base 15 by a drive force of a motor (here, the motor includes the permanent magnets 12 and 13 and the coil part 16).

The rotation body 14 is rotatably supported by bearing portions T1 and T2 (here, the rotary electric connectors 17 and 18) at both sides of the support base 15. The rotation body 14 has, for example, a cylindrical shape and the permanent magnets 12 and 13 are disposed on the cylindrical inner peripheral surface to face each other with a hollow region interposed therebetween. Further, the coil part 16 is disposed in the cylindrical hollow region of the rotation body 14.

The permanent magnets 12 and 13 are disposed so that both facing surfaces have opposite polarities, that is, one permanent magnet faces an N pole inside the cylindrical hollow region and the other permanent magnet faces an S pole inside the cylindrical hollow region.

The support base 15 rotatably supports the rotation body 14. The support base 15 is disposed to sandwich the rotation body 14 from both sides while being fixed to the casing of the ultrasonic probe 10. The rotary electric connectors 17 and 18 are respectively disposed at the bearing portions T1 and T2 on both sides of the support base 15 and the bearing portions T1 and T2 according to the embodiment are formed as the rotary electric connectors 17 and 18. Here, the bearing portions T1 and T2 may be additionally provided with other bearing mechanisms other than the rotary electric connectors 17 and 18.

The coil part 16 includes a shaft 16a which is bonded to the bearing portions T1 and T2 on both sides of the support base 15 and a coil 16b for three phases (U, V, and W phases) wound on the shaft 16a. Then, the three-phase coil 16b is wound by shifting the positions around the shaft 16a in the extension direction and motor drive wires are respectively connected to the three-phase coils. Then, the three-phase coil 16b forms a rotation magnetic field around the shaft 16a in the extension direction by the three-phase AC power supplied thereto. In addition, the three-phase coil 16b is disposed to face the permanent magnets 12 and 13 with a gap interposed therebetween and applies a rotation magnetic field to the permanent magnets 12 and 13.

Further, the supply of the three-phase AC power to the coil part 16 is controlled by, for example, the control unit 21 of the main body 20. The control unit 21 servo-controls the rotation body 14 based on, for example, a sensor signal from the sensor unit 19.

The sensor unit 19 detects the rotation position of the rotation body 14. The sensor unit 19 is, for example, a rotary encoder and detects the rotation position of the rotation body 14 by detecting a magnetic field generated by a permanent magnet (not illustrated) for a sensor disposed on the rotation body 14 using a Hall element. The sensor signal of the sensor unit 19 is transmitted to, for example, the control unit 21 of the main body 20.

As described above, in the ultrasonic probe 10 according to the embodiment, an outer rotor rotation type brushless DC motor which rotates the rotation body 14 includes the permanent magnets 12 and 13 and the coil part 16. Here, a mechanism which rotates the rotation body 14 may be of an arbitrary drive type.

The rotary electric connectors 17 and 18 are connectors for exchanging electric signals with the ultrasonic oscillator 11 disposed on the rotation body 14. Further, the rotary electric connectors 17 and 18 according to the embodiment rotatably support the rotation body 14 in the bearing portions T1 and T2 on both sides of the support base 15.

Further, the left rotary electric connector 17 of FIG. 4 is provided to exchange electric signals between the ultrasonic oscillator 11 and the transmitting/receiving unit 22 of the main body 20 and the right rotary electric connector 18 of FIG. 4 is provided to ensure the ground of the ultrasonic oscillator 11. Since the rotary electric connector 17 and the rotary electric connector 18 have the same configuration, only the rotary electric connector 17 will be described below.

Here, the ground of the ultrasonic oscillator 11 may be formed at the rotation body 14 itself and in that case, the rotary electric connector 18 is not necessary. In that case, a bearing may be disposed at the bearing portion T1 of the support base 15 instead of the rotary electric connector 18. Meanwhile, a bearing may be provided at the bearing portions T1 and T2 on both sides of the support base 15 along with the rotary electric connectors 17 and 18 in order to smoothly rotate the rotation body 14.

[Configuration of Rotary Electric Connector]

Next, a configuration of the rotary electric connector 17 according to the embodiment and a structure in which the ultrasonic oscillator 11 exchange electric signals with the main body 20 using the rotary electric connector 17 will be described with reference to FIG. 5 to FIG. 7.

FIG. 5 is a diagram showing an example of a configuration of the rotary electric connector 17. FIG. 6 is a cross-sectional view taken along a line L-L′ of FIG. 5. FIG. 7 is a diagram showing a rotary contact member 17c of the rotary electric connector 17.

Hereinafter, a description will be made on the assumption that a coordinate axis which connects the rotation center position of the rotation body 14 in a linear shape will be referred to as a “rotation axis” and a direction in which the rotation body 14 rotates about the rotation axis will be referred to as a “rotation axis rotation M”.

The rotary electric connector 17 includes the rotary electrode 17a, the fixed electrode 17b, and the rotary contact member 17c.

The rotary electrode 17a is an electrode member that is bonded to the rotation body 14 so that its rotation axis is the same as the rotation axis of the rotation body 14 and rotates along with the rotation body 14. The rotary electrode 17a is formed in, for example, a cylindrical shape and is bonded to a disc surface at one end side of the cylindrical rotation body 14.

The fixed electrode 17b is an electrode member that is bonded to the support base 15. The fixed electrode 17b is disposed inside a hollow region of the rotary electrode 17a. The fixed electrode 17b is formed in, for example, a cylindrical shape having a diameter smaller than the diameter of the inner periphery of the rotary electrode 17a.

In other words, in the rotary electric connectors 17, one rotary electric connector is a center member disposed at the inside in the radial direction in the rotation axis rotation M of the rotation body 14, the other rotary electric connector is an outer peripheral member disposed at the outside in the radial direction in the rotation axis rotation M of the rotation body 14, and the outer peripheral member at the other side surrounds the outer peripheral surface of the center member at one side. Additionally, in the embodiment, the fixed electrode 17b is set as the center member and the rotary electrode 17a is set as the outer peripheral member. In contrast, the fixed electrode 17b may be set as the outer peripheral member and the rotary electrode 17a may be set as the center member.

The outer peripheral surface of the fixed electrode 17b and the inner peripheral surface of the rotary electrode 17a face each other while being separated from each other to form an annular guide groove A in the rotation axis rotation M. Then, the rotary contact member 17c which electrically connects the rotary electrode 17a and the fixed electrode 17b to each other is disposed inside the annular guide groove A.

The rotary contact member 17c exchanges electric signals between the rotary electrode 17a and the fixed electrode 17b. The rotary contact member 17c is an electrode member having a columnar, spherical, or conical shape (in the embodiment, a columnar shape) and the rotary electrode 17a and the fixed electrode 17b are disposed in the annular guide groove A to rotate while contacting each other. In other words, the rotary contact member 17c serves as a rolling body of a roller bearing between the rotary electrode 17a and the fixed electrode 17b. In addition, the rotary contact member 17c is formed so that its diameter is larger than a groove width of the annular guide groove A.

The rotary contact member 17c according to the embodiment is formed by applying a conductive metal material onto a surface of an elastically deformable member (for example, a rubber member). Here, the rotary contact member 17c may be formed of an elastic conductive material (for example, a rubber member having a conductive particle). In this way, the rotary contact member 17c is formed as the elastic member. Thus, at the time of rotating the rotary electrode 17a, the rotary contact member 17c rolls while being elastically deformed inside the annular guide groove A to contact both the rotary electrode 17a and the fixed electrode 17b at all times. Accordingly, the rotary contact member 17c can ensure a large contact area with the rotary electrode 17a and the fixed electrode 17b.

Further, in the embodiment, a plurality of the rotary contact members 17c (in the embodiment, eight rotary contact members 17c) are laid in the annular guide groove A to be adjacent to each other.

If the rotary electric connector 17 includes only one rotary contact member 17c, the rotary contact member 17c is elastically deformed when rolling in the annular guide groove A. Accordingly, since the rotation axis of the rotation body 14 is displaced vertically and laterally, there is concern that the ultrasonic wave transmitting/receiving direction of the ultrasonic oscillator 11 may change. In this regard, since the plurality of rotary contact members 17c are disposed in the guide groove A, the rotary electrode 17a are urged toward the reference position of the fixed electrode 17b, that is, the reference position of the rotation axis of the rotation body 14 at all times by the elastic force of the plurality of rotary contact members 17c. As a result, the displacement of the rotation axis is suppressed.

Further, the rotary electrode 17a is connected to the ultrasonic oscillator 11 via the signal wire 17d and the fixed electrode 17b is connected to the main body 20 via the lead wire 17e. That is, an electric signal which is generated by the ultrasonic oscillator 11 is transmitted to the main body 20 via the signal wire 17d, the rotary electrode 17a, the rotary contact member 17c, the fixed electrode 17b, and the lead wire 17e in this order (which is indicated by a dotted line in FIG. 5). On the contrary, the electric signal of the main body 20 is transmitted to the ultrasonic oscillator 11 via the lead wire 17e, the fixed electrode 17b, the rotary contact member 17c, the rotary electrode 17a, and the signal wire 17d in this order.

The rotary electric connector 17 is operated as below when the rotation body 14 rotates.

In the rotary electric connector 17, the rotary electrode 17a first rotates along with the rotation of the rotation body 14 at the time of rotating the rotation body 14. Then, the plurality of rotary contact members 17c revolve along the annular guide groove A while rotating at the corresponding position by the friction applied from the contact portion with the rotary electrode 17a in response to the rotation of the rotary electrode 17a. In other words, the plurality of rotary contact members 17c roll in the annular guide groove A to move in a planetary motion.

At this time, the plurality of rotary contact members 17c roll while urging the rotary electrode 17a toward the reference position of the rotation axis in an elastically deformed state so as to contact both electrode members of the rotary electrode 17a and the fixed electrode 17b at all times. Thus, the rotary electrode 17a and the fixed electrode 17b are electrically connected to each other at all times even when the rotation body 14 rotates. In addition, the rotation body 14 can rotate while the displacement of the rotation axis is suppressed.

As described above, according to the ultrasonic probe 10 of the embodiment, it is possible to reliably exchange electric signals with the ultrasonic oscillator 11 while stably keeping the ultrasonic wave transmitting/receiving direction of the ultrasonic oscillator 11 using the rotary electric connector 17 to exchange electric signals with the ultrasonic oscillator 11. Accordingly, since noises superimposed on the receiving signals of the ultrasonic echo decrease, the image quality of the ultrasonic image can be improved.

Further, since the rotary electric connector 17 according to the embodiment does not cause the abrasion of the members (for example, the rotary electrode 17a, the fixed electrode 17b, and the rotary contact member 17c) compared to an electric connection structure according to the related art using the brush and the slip ring, it is possible to suppress a change in electric connection state over time.

### Second Embodiment

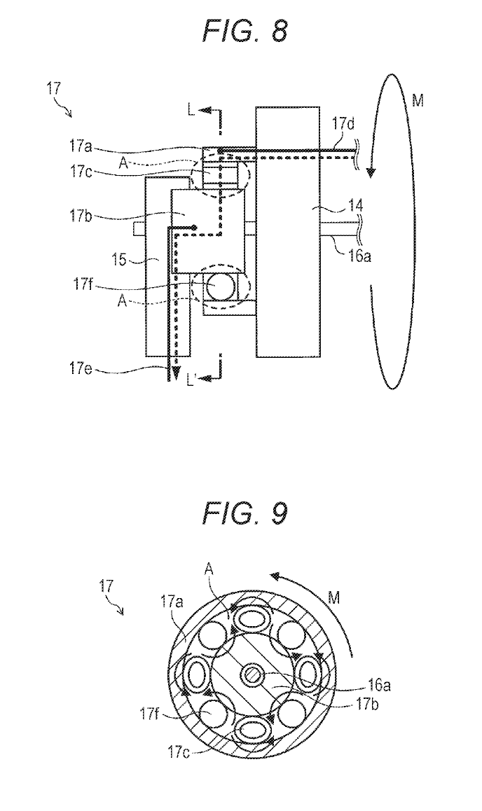

Hereinafter, an example of a configuration of an ultrasonic probe 10 according to a second embodiment will be described with reference to FIG. 8 and FIG. 9.

The ultrasonic probe 10 according to the embodiment is different from that of the first embodiment in that a plurality of high-rigid rolling bodies 17f are disposed in the annular guide groove A of the rotary electric connector 17 along with the rotary contact member 17c. In addition, a description of the same configuration as that of the first embodiment will be omitted (hereinafter, the same applies to other embodiments).

FIG. 8 and FIG. 9 are diagrams (a side view and a cross-sectional view of the rotary electric connector 17) respectively corresponding to FIG. 5 and FIG. 6 of the first embodiment and showing an example of a configuration of the rotary electric connector 17 according to the embodiment.

As described above, since the rotary contact member 17c is elastically deformable, the rotary contact member 17c is elastically deformed when rolling in the annular guide groove A. Accordingly, there is concern that the rotation axis of the rotation body 14 is displaced vertically and laterally.

In the rotary electric connector 17 according to the embodiment, a plurality of high-rigid rolling bodies 17f are disposed in the annular guide groove A along with the rotary contact member 17c in order to improve the concentricity of the rotation axis of the rotation body 14.

Since the high-rigid rolling body 17f can be formed as a columnar, spherical, or conical member similarly to the rotary contact member 17c, a metal ball substantially having the same diameter as the diameter (here, the diameter of the column) of the rotary contact member 17c is typically used. In addition, the high-rigid rolling body 17f does not need to be formed of a metal material as long as the rigidity is higher than that of the rotary contact member 17c.

The plurality of high-rigid rolling bodies 17f contact the rotary electrode 17a and the fixed electrode 17b at a plurality of positions of the annular guide groove A and cause a reaction force against a force in which the rotary electrode 17a is displaced toward the outside from the reference position of the fixed electrode 17b, that is, the reference position of the rotation axis of the rotation body 14. As a result, it is possible to further suppress the displacement of the rotation axis of the rotation body 14.

More desirably, the high-rigid rolling bodies 17f are alternately arranged in the annular guide groove A along with the rotary contact members 17c as in FIG. 9. Accordingly, since it is possible to suppress the displacement of the rotation axis of the rotation body 14 and to prevent the contact between the rotary contact members 17c, it is possible to also suppress the abrasion of the rotary contact member 17c. Additionally, FIG. 9 shows a state where four high-rigid rolling bodies 17f are alternately arranged in the annular guide groove A along with four rotary contact members 17c.

More desirably, the diameter of the high-rigid rolling body 17f is set to be substantially the same as the diameter (here, the diameter of the column) of the rotary contact member 17c and is more desirably slightly smaller than the diameter of the rotary contact member 17c. Accordingly, it is possible to reliably keep a state where the rotary contact member 17c contacts both the rotary electrode 17a and the fixed electrode 17b.

As described above, when the high-rigid rolling bodies 17f are disposed in the annular guide groove A of the rotary electric connector 17 along with the rotary contact members 17c as in the ultrasonic probe 10 according to the embodiment, it is possible to further suppress the displacement of the rotation axis of the rotation body 14.

### Third Embodiment

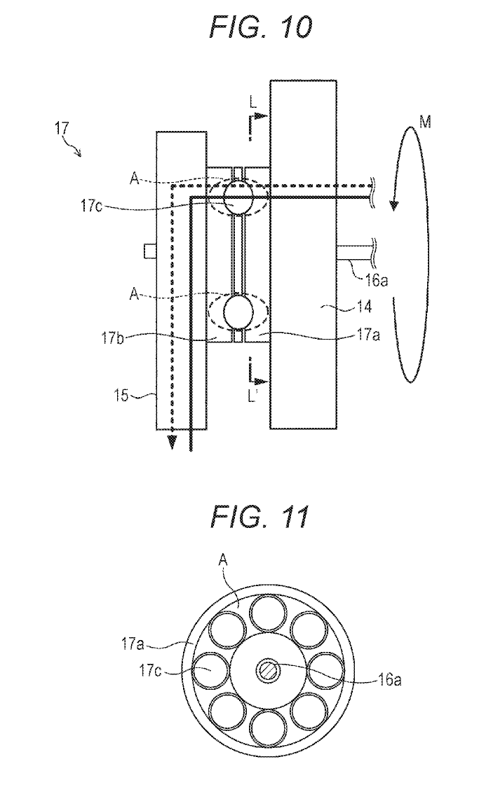

Hereinafter, an example of a configuration of an ultrasonic probe 10 according to a third embodiment will be described with reference to FIG. 10 to FIG. 12.

The ultrasonic probe 10 according to the embodiment is different from that of the first embodiment in that the rotary electrode 17a and the fixed electrode 17b are disposed to face each other in the extension direction of the rotation axis and a track base 17g rotatably supporting the rotary contact member 17c is disposed in a region in which the rotary electrode 17a and the fixed electrode 17b face each other.

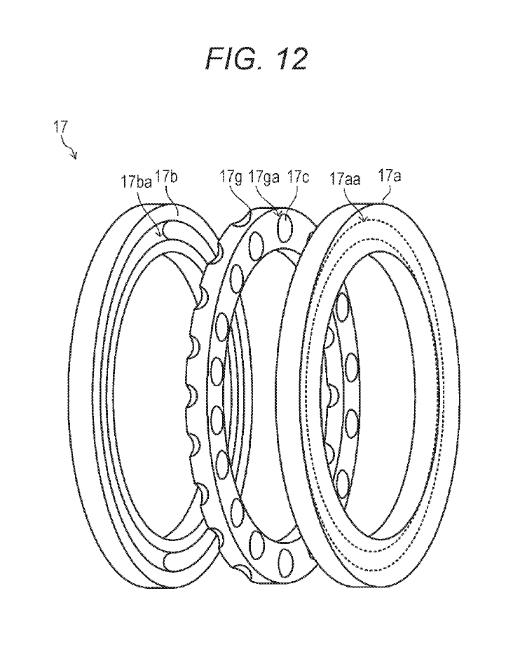

FIG. 10 and FIG. 11 are diagrams (a side view and a cross-sectional view of the rotary electric connector 17) respectively corresponding to FIG. 5 and FIG. 6 of the first embodiment and are diagrams showing an example of a configuration of the rotary electric connector 17 according to the embodiment. FIG. 12 is an exploded diagram showing the rotary electric connector 17 according to the embodiment.

The rotary electric connector 17 according to the embodiment is formed as a thrust ball bearing.

More specifically, both the rotary electrode 17a and the fixed electrode 17b according to the embodiment are formed as plate-shaped members having annular track grooves 17aa and 17ba. Then, since the rotary electrode 17a and the fixed electrode 17b are disposed in the extension direction of the rotation axis so that the annular track grooves 17aa and 17ba face each other, the guide groove A on which the rotary contact member 17c rolls is formed.

Then, in the rotary electric connector 17 according to the embodiment, the track base 17g which regulates the rotation state of the rotary contact member 17c is disposed in a region in which the rotary electrode 17a and the fixed electrode 17b face each other.

The track base 17g is formed in an annular shape along the guide groove A. Further, the track base 17g includes a plurality of fitting grooves 17ga on which the rotary contact member 17c is disposed and is disposed along the guide groove A while the rotary contact member 17c is disposed in the fitting groove 17ga.

With such a configuration, the track base 17g is supported in a region in which the rotary electrode 17a and the fixed electrode 17b face each other by the rotary contact member 17c without contacting the rotary electrode 17a and the fixed electrode 17b. Then, the track base 17g performs the rotation axis rotation M in response to the rotation of the rotary contact member 17c. Further, since the plurality of rotary contact members 17c are respectively disposed in the fitting grooves 17ga of the track base 17g, the plurality of rotary contact members rotate along the guide groove A without contacting other rotary contact members 17c.

As described above, since the track base 17g is disposed as in the ultrasonic probe 10 according to the embodiment, it is possible to realize a more stable exchange of electric signals between the ultrasonic oscillator 11 and the main body 20 by stabilizing the rotation state of the rotary contact member 17c. In addition, since the track base 17g is disposed in the guide groove A, it is possible to prevent a problem in which the rotary contact member 17c is separated from the guide groove A.

Further, FIG. 10 to FIG. 12 show a case where the rotary contact member 17c has a spherical shape, but the rotary contact member may be, of course, formed in a columnar shape similarly to the first embodiment.

Similarly to the first embodiment, in the track bases 17g, one member may be a center member disposed at the inside in the radial direction of the rotation body 14 in the rotation axis rotation M and the other member may be an outer peripheral member disposed at the outside in the radial direction of the rotation body 14 in the rotation axis rotation M.

### Fourth Embodiment

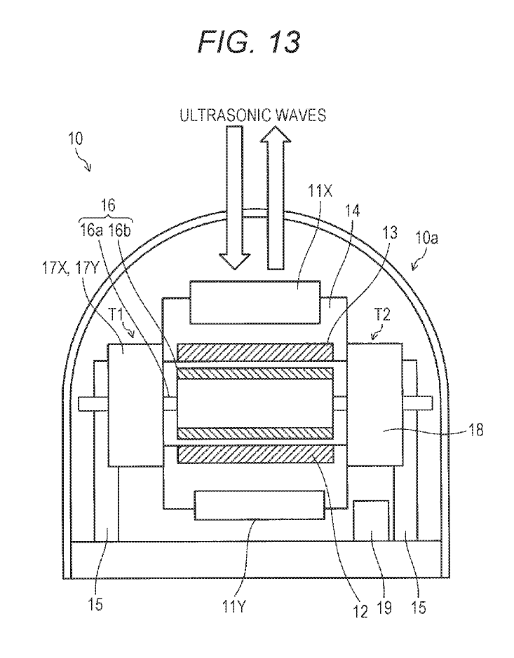

Hereinafter, an example of a configuration of an ultrasonic probe 10 according to a fourth embodiment will be described with reference to FIG. 13 to FIG. 15.

The ultrasonic probe 10 according to the embodiment is different from that of the first embodiment in that two sets of rotary electric connectors 17 (referred to as a “first rotary electric connector 17X” and a “second rotary electric connector 17Y”) which are insulated and separated from each other are disposed at the inside and the outside in the radial direction of the rotation body 14 in the rotation axis rotation.

FIG. 13 is a diagram corresponding to FIG. 4 of the first embodiment and is a diagram showing an example of a configuration of the ultrasonic probe 10 according to the embodiment.

In the ultrasonic probe 10 according to the embodiment, two ultrasonic oscillators 11 (referred to as a “first ultrasonic oscillator 11X” and a “second ultrasonic oscillator 11Y”) are disposed on the outer peripheral surface of the rotation body 14.

Here, the first ultrasonic oscillator 11X and the second ultrasonic oscillator 11Y have different signal transmission paths at the time of exchanging electric signals with the main body 20. Then, the ultrasonic probe 10 according to the embodiment include two sets of rotary electric connectors 17 (the first rotary electric connector 17X and the second rotary electric connector 17Y) having different electric signal channels so as to individually exchange electric signals with the first ultrasonic oscillator 11X and the second ultrasonic oscillator 11Y.

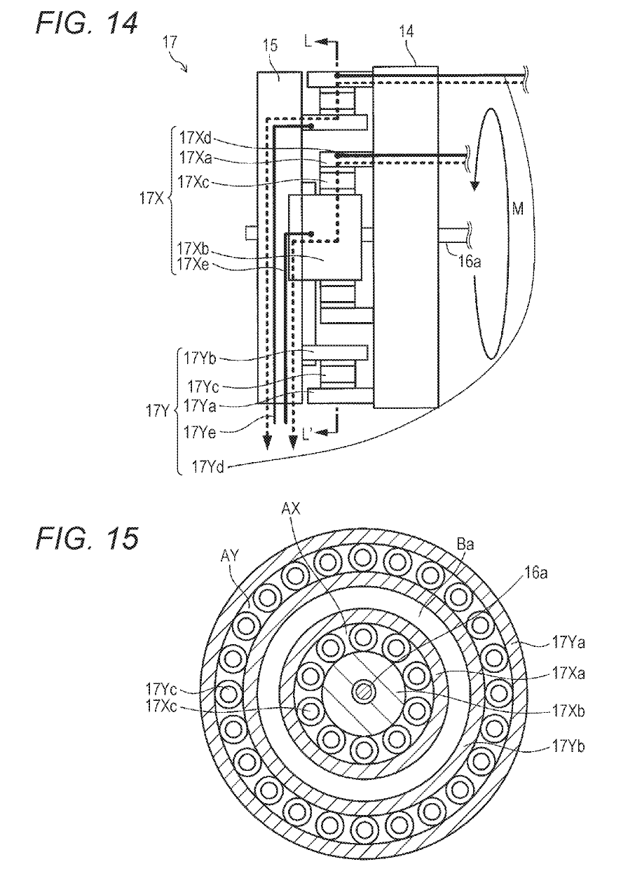

FIG. 14 and FIG. 15 are diagrams (a side view and a cross-sectional view of the rotary electric connector 17) respectively corresponding to FIG. 5 and FIG. 6 of the first embodiment and showing an example of a configuration of the rotary electric connector 17 according to the embodiment.

The first rotary electric connector 17X is formed at the inner peripheral side of the rotation axis rotation M and the second rotary electric connector 17Y is formed at the outer peripheral side of the rotation axis rotation M.

More specifically, the first rotary electric connector 17X includes a first rotary electrode 17Xa, a first fixed electrode 17Xb, a first rotary contact member 17Xc disposed in a guide groove AX formed by the first rotary electrode 17Xa and the first fixed electrode 17Xb, a first rotation-side signal wire 17Xd, and a first fixed side lead wire 17Xe and constitutes the same rotary electric connector as the rotary electric connector 17 of the first embodiment by these members.

Further, the second rotary electric connector 17Y includes a second rotary electrode 17Ya, a second fixed electrode 17Yb, a second rotary contact member 17Yc disposed in a guide groove AY formed by the second rotary electrode 17Ya and the second fixed electrode 17Yb, a second rotation-side signal wire 17Yd, and a second fixed side lead wire 17Ye and constitutes the same rotary electric connector as the rotary electric connector 17 of the first embodiment by these members.

The second rotary electrode 17Ya and the second fixed electrode 17Yb of the second rotary electric connector 17Y are disposed at the outside in the radial direction of the rotation axis rotation M in relation to the first rotary electrode 17Xa and the first fixed electrode 17Xb of the first rotary electric connector 17X. Then, the first rotary electric connector 17X and the second rotary electric connector 17Y are insulated and separated from each other by a space Ba therebetween.

As described above, according to the ultrasonic probe 10 of the embodiment, since a plurality of signal channels can be realized by two sets of rotary electric connectors 17X and 17Y, it is possible to individually exchange electric signals with the plurality of ultrasonic oscillators 11X and 11Y.

Further, three sets or more of the rotary electric connectors 17X and 17Y may be, of course, disposed in the radial direction of the rotation body 14 in the rotation axis rotation.

### Fifth Embodiment

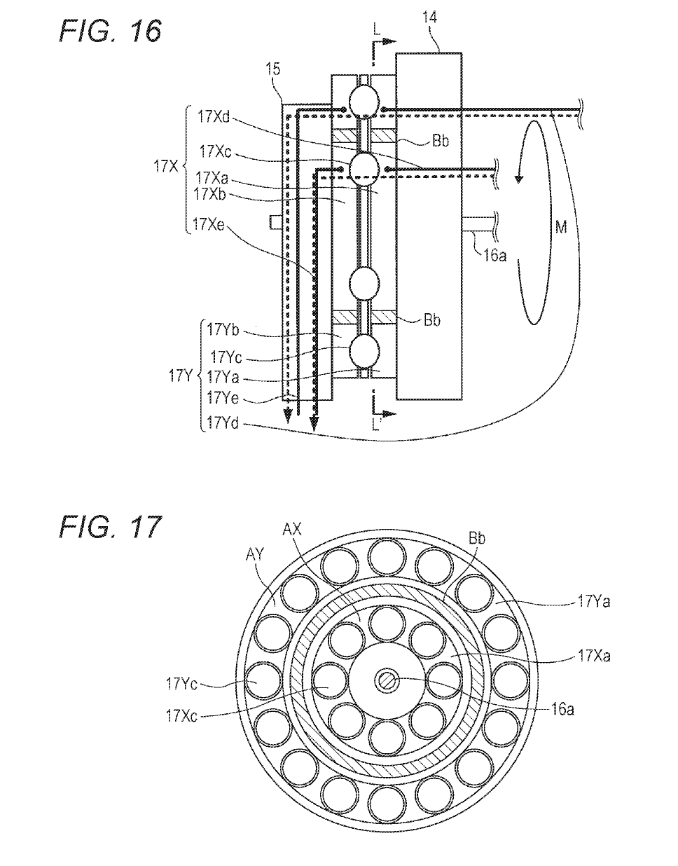

Hereinafter, an example of a configuration of an ultrasonic probe 10 according to a fifth embodiment will be described with reference to FIG. 16 and FIG. 17.

The ultrasonic probe 10 according to the embodiment is different from that of the fourth embodiment in that the rotary electric connector according to the third embodiment is adopted to constitute two sets of rotary electric connectors 17X and 17Y.

FIG. 16 and FIG. 17 are diagrams (a side view and a cross-sectional view of the rotary electric connector 17) respectively corresponding to FIG. 14 and FIG. 15 of the fourth embodiment and are diagrams showing an example of a configuration of the rotary electric connector 17 according to the embodiment.

Similarly to the ultrasonic probe 10 according to the fourth embodiment, the ultrasonic probe 10 according to the embodiment also includes two sets of the first rotary electric connector 17X and the second rotary electric connector 17Y having different electric signal channels and provided in the bearing portion T1 of the rotation body 14 of the support base 15 in order to individually exchange electric signals with two ultrasonic oscillators 11.

The first rotary electric connector 17X is formed at the inner peripheral side of the rotation axis rotation M and the second rotary electric connector 17Y is formed at the outer peripheral side of the rotation axis rotation M.

More specifically, the first rotary electric connector 17X includes the first rotary electrode 17Xa, the first fixed electrode 17Xb, the first rotary contact member 17Xc disposed in a guide groove Aix formed by the first rotary electrode 17Xa and the first fixed electrode 17Xb, the first rotation-side signal wire 17Xd, and the first fixed side lead wire 17Xe and constitutes the same rotary electric connector as the rotary electric connector 17 of the first embodiment by these members.

Further, the second rotary electric connector 17Y includes the second rotary electrode 17Ya, the second fixed electrode 17Yb, the second rotary contact member 17Yc disposed in the guide groove AY formed by the second rotary electrode 17Ya and the second fixed electrode 17Yb, the second rotation-side signal wire 17Yd, and the second fixed side lead wire 17Ye and constitutes the same rotary electric connector as the rotary electric connector 17 of the first embodiment by these members.

The second rotary electrode 17Ya and the second fixed electrode 17Yb of the second rotary electric connector 17Y are disposed at the outside in the radial direction in the rotation axis rotation M in relation to the first rotary electrode 17Xa and the first fixed electrode 17Xb of the first rotary electric connector 17X. Then, the first rotary electric connector 17X and the second rotary electric connector 17Y are insulated and separated by an insulation region Bb therebetween.

As described above, according to the ultrasonic probe 10 of the embodiment, since it is possible to realize a plurality of signal channels by the rotary electric connectors 17X and 17Y, it is possible to individually exchange electric signals with the plurality of ultrasonic oscillators 11.

### Other Embodiments

The present invention is not limited to the above-described embodiments and various modifications can be considered.

In the above-described embodiments, an example of a configuration of the rotary electric connector 17 is shown variously. Here, various combinations of configurations in the embodiments may be used.

Further, in the above-described embodiments, as an example of a configuration of the rotary electric connector 17, electric signals are exchanged between the ultrasonic oscillator 11 and the main body 20 using the lead wire 17e drawn to the main body 20. Here, the lead wire 17e may be, of course, connected to a signal amplification relay circuit board disposed in the ultrasonic probe 10.

Meanwhile, in the ultrasonic probes 10 according to the third embodiment and the fifth embodiment, the rotary electrode 17a and the fixed electrode 17b are disposed to face each other in the extension direction of the rotation axis, but from the viewpoint of preventing the separation of the rotary contact member 17c, a configuration in which the rotary electrode 17a and the fixed electrode 17b are disposed at the inside and the outside of the rotation axis in the radial direction to face each other as in the first embodiment is more desirable.

According to the ultrasonic probe of the present disclosure, it is possible to reliably exchange electric signals with the ultrasonic oscillator while rotating the ultrasonic oscillator.

Although embodiments of the present invention have been described and illustrated in detail, the disclosed embodiments are made for purposes of illustration and example only and not limitation. The scope of the present invention should be interpreted by terms of the appended claims. The technology described in the claims includes various modifications and changes of the above-described embodiments.

What is claimed is:

1. An ultrasonic probe comprising:
a support base;
a rotation body which rotates while being supported by the support base;
an ultrasonic oscillator which is disposed on an outer peripheral surface of the rotation body; and
a rotary electric connector which is disposed to axially support the rotation body by the support base and exchanges electric signals with the ultrasonic oscillator,
wherein the rotary electric connector includes:
a rotary electrode which is bonded to the rotation body and rotates along with the rotation body,
a fixed electrode which is bonded to the support base to face the rotary electrode and forms an annular guide groove in a rotation axis rotation in a region facing the rotary electrode, and
a rotary contact member that is disposed inside the guide groove and rolls along the guide groove while contacting both the rotary electrode and the fixed electrode in response to the rotation of the rotary electrode.

2. The ultrasonic probe according to claim 1,
wherein in the rotary electrode and the fixed electrode of the rotary electric connector, one electrode is a center member disposed at the inside in the radial direction of the rotation body in the rotation axis rotation and the other electrode is an outer peripheral member disposed at the outside in the radial direction of the rotation body in the rotation axis rotation, and
the outer peripheral member corresponding to the other electrode is disposed to surround the outer peripheral surface of the center member corresponding to one electrode.

3. The ultrasonic probe according to claim 1,
wherein the rotary electrode and the fixed electrode of the rotary electric connector are all plate-shaped members having an annular track groove and are disposed to form the guide groove by allowing the track grooves to face each other along the extension direction of the rotation axis of the rotation body.

4. The ultrasonic probe according to claim 1,
wherein the rotary contact member has a spherical, columnar, or conical shape.

5. The ultrasonic probe according to claim 1,
wherein a plurality of the rotary contact members are disposed inside the guide groove.

6. The ultrasonic probe according to claim 1,
wherein the rotary contact member is formed as an elastically deformable member.

7. The ultrasonic probe according to claim 1,
wherein a plurality of high-rigid rolling bodies having a spherical shape, a columnar shape, or a conical shape are disposed inside the guide groove along with the rotary contact member.

8. The ultrasonic probe according to claim 1,
wherein a track base having an annular shape along the guide groove and rotatably supporting the rotary contact member is disposed in a region in which the rotary electrode and the fixed electrode face each other.

9. The ultrasonic probe according to claim 1,
wherein at least two sets of ultrasonic oscillators are disposed on the outer peripheral surface of the rotation body, and
at least two sets of the rotary electric connectors which are insulated and separated from each other are disposed at the inside and the outside in the radial direction of the rotation body in the rotation axis rotation to exchange electric signals with two sets of the ultrasonic oscillators.

10. The ultrasonic probe according to claim 1,
wherein the rotation body includes a permanent magnet, and
the support base includes a coil part which generates a rotation force in the rotation body by applying a rotation magnetic field to the permanent magnet of the rotation body.

11. The ultrasonic probe according to claim 1,
wherein the support base, the rotation body, the ultrasonic oscillator, and the rotary electric connector are disposed inside a window portion which keeps a coupling liquid in a liquid-tight state.

12. An ultrasonic diagnostic apparatus comprising:
the ultrasonic probe according to claim 1.
